(19) United States
(12) Patent Application Publication
(10) Pub. No.: US 2007/0128128 A1
(43) Pub. Date: Jun. 7, 2007

(54) Method for the innoformulation of a biocompatible galenic base

(75) Inventors: Jean-Noel Thorel, Paris (FR); Gerard Redziniak, Antony (FR)

Correspondence Address: OLIFF & BERRIDGE, PLC, P.O. BOX 19928, ALEXANDRIA, VA, 22320, US

(73) Assignee: Jean-Noel Thorel, Paris (FR)

(21) Appl. No.: 10/583,892

(22) PCT Filed: Dec. 23, 2004
(86) PCT No.: PCT/FR04/03376

§ 371 Date: Oct. 23, 2006

(30) Foreign Application Priority Data
Dec. 23, 2003 (FR) 0315290

(51) Int. Cl. A61K 8/73

(52) U.S. Cl. 424/49; 424/70.13

(57) ABSTRACT

A high-tolerance biocompatible and bioregulatory dermic and/or cosmetic galenic base contains, in the aqueous phase thereof, at least two polyols selected from osides, oses or reduction products of oses, wherein at least two of the polyols are selected from reduction products of oses that are mannitol and xylitol. A dermic and/or cosmetic galenic base includes a fatty phase including a substance selected from lipo-regulatory substances. Polyols selected from osides, oses or reduction products of oses in the aqueous phase of a dermic and/or cosmetic base improve the tolerance thereof and optimize the active effects thereof. A cosmetic and/or dermo-cosmetic composition includes a base according to the invention.

The tolerability and harmlessness of products created for living beings, their environment and their safety in use are more topical than ever, irrespective of the field. In the matter of cosmetic formulation and even more in the field of dermo-cosmetic formulations, it is essential to take these factors into account. By dint of their intended purpose, dermo-cosmetic products are often applied mainly to delicate and reactive skin. The obligation of neutrality is therefore even more important than for conventional cosmetic products like care or make-up products.

In fact, the general biological consequence of skin diseases is a weakening of the cutaneous ecosystem and an increase in the sensitivity of the skin to external agents. Certain elements present in cosmetic bases, especially surfactants, preservatives and the quality of the water used, play a part in the problems of reactivity or dysfunction associated with these external agents.

In a cosmetic and/or dermo-cosmetic formulation intended for developing an emulsion, a cream, a milk, a lotion or an oil, it is conventional to make a distinction between two elements, namely on the one hand the base or support, referred to as galenic (subject of the present invention), and on the other hand the cosmetic, dermo-cosmetic or medicinal active ingredients.

In the state of the art, the dermal and/or cosmetic galenic base has no biological vocation and only constitutes a vehicle or support intended for:

carrying the active ingredient to the right place,

providing the user or consumer with the sensory effects he expects in relation to its intended purpose (eyes, body, face), its form (milk, cream) and its perfume,

stabilizing and preserving the cosmetic product and the appropriate active ingredients, and

assuring the expected basic function(s) while guaranteeing the greatest possible compatibility with all types of products and active ingredient complexes.

It must also have an excellent tolerability on the skin.

In cosmetics and/or dermo-cosmetics, the bases mainly consist of two phases, namely an aqueous phase and a fatty phase, to which emulsifiers, technical stabilizers and preservatives and cosmetic or pharmaceutical active ingredients are added.

These phases can be single and continuous, continuous aqueous phase, lotion, continuous fatty phase, oil or in a mixture, two-phase lotion, foam, emulsion multiple creams, foams.

When applied to a delicate and/or reactive skin, this base itself can sometimes intrinsically cause irritation reactions and skin intolerance phenomena which are in addition to the reactions caused by the active ingredients or are due to the pathological skin conditions already present.

The galenic base used as vehicle therefore has to be perfectly tolerated, irrespective of the state of the skin and the active ingredients carried.

The present invention makes it possible to solve all the problems referred to above, and relates to a dermal and/or cosmetic galenic base of very high tolerability which is perfectly tolerated by the skin, irrespective of the active ingredients and the customary additives used in cosmetics and/or dermo-cosmetics which are incorporated therein, and irrespective of the pathological skin conditions.

This base is defined so as to respect the ecosystem of the skin and be biocompatible with the cosmetic and/or medicinal active ingredients and with the biological state of skin rendered delicate by disease.

By improving cell viability in particular, this dermal and/or cosmetic galenic base makes it possible to obtain an optimal increased resistance to external agents and an optimal hydration, and hence a less reactive skin. It also allows a reduction in allergenic phenomena while containing reduced amounts of preservatives, which can also be a source of intolerance reactions.

FR2609309, EP1354580 and J20060893 have disclosed the use of polyols or oses as cosmetic active principles, e.g. as cell stimulating substances, energy sources or antioxidants, but polyols have never been described as constituents of a dermal and/or cosmetic galenic base.

The present invention relates to a dermal and/or cosmetic galenic base, characterized in that its aqueous phase contains at least two polyols each selected from the group comprising osides, oses and ose reduction products.

It further relates to a dermal and/or cosmetic galenic base whose aqueous phase contains at least two polyols each selected from the group comprising osides, oses and ose reduction products, and which is characterized in that at least two of these polyols are selected from the group of ose reduction products comprising mannitol and xylitol.

According to the invention, the dermal and/or cosmetic galenic base can also be characterized in that one polyol is selected from the group of oses comprising glucose, rhamnose, xylose, mannose and fructose.

It relates more particularly to a dermal and/or cosmetic galenic base according to the invention, characterized in that the polyol is selected from the group of oses comprising glucose, rhamnose, xylose, mannose and fructose.

In one embodiment, the dermal and/or cosmetic galenic base according to the invention is characterized in that one polyol selected from the group of oses is rhamnose.

It relates more particularly to a dermal and/or cosmetic galenic base according to the invention, characterized in that the polyol is selected from the group of ose reduction products comprising mannitol and xylitol.

It relates more particularly to a dermal and/or cosmetic galenic base according to the invention, characterized in that the polyol is selected from the group of osides comprising fructooligosaccharides, the trisaccharide polymer of α-L-fucose-1->3-α-D-galactose-1->3-α-D-galacturonic acid, hyaluronic acid, chondroitin sulfate, cyclodextrins, galactoarabinan and insulin.

The aqueous phase according to the invention also makes it possible to improve the cell viability of a culture of fibroblasts and keratinocytes, compared with a conventional aqueous phase.

In one embodiment, the aqueous phase of the dermal and/or cosmetic galenic base comprises at least one polyol selected from the group comprising oses and osides, and at least one polyol selected from the group comprising ose reduction products.

In another embodiment, the aqueous phase of the dermal and/or cosmetic galenic base comprises at least one polyol selected from the group comprising oses and osides, and at least two polyols selected from the group comprising ose reduction products.

As specified above, some cosmetic, dermo-cosmetic and/or care formulations contain a fatty phase or consist solely of a fatty phase.

Some polyols may not be soluble in a fatty phase or may be so poorly soluble that they are unable to play their role as ingredients of this phase, in which case said polyols can be chemically modified, e.g. by the chemical grafting of a liposoluble chain or by polymerization, for example in order to obtain products like Rhamnosoft®, which is a polymer obtained by the fermentation of sorbitol (INCI nomenclature: biosaccharide gum-2).

The invention therefore further relates to a dermal and/or cosmetic galenic base whose fatty phase contains at least two liposoluble polyols each selected from the group comprising osides, oses, ose reduction products and chemically modified oses.

It relates in particular to a galenic base, characterized in that the chemically modified ose is selected from the group comprising Rhamnosoft®, cetearyl glucoside, mannitan laurate and glucose glutamate.

Furthermore, this fatty phase can sometimes intrinsically have adverse effects on the skin, especially due to the action of the lipoperoxides which may form there. Thus, to guarantee the high tolerability maintained by the aqueous phase, a so-called liporegulatory substance is added when the formulation contains a fatty phase.

This liporegulatory substance makes it possible to re-equilibrate the lipid elements of the skin and optimize the tolerability on deficient skin.

By becoming incorporated in the cutaneous molecular structures (cell membranes, epidermal intercellular cement), this liporegulatory substance lowers the reactivity threshold of the skin.

The invention thus relates to a dermal and/or cosmetic galenic base according to the invention, characterized in that it also contains a fatty phase comprising a substance selected from liporegulatory substances.

It relates more particularly to a dermal and/or cosmetic galenic base according to the invention, characterized in that the liporegulatory substance is selected from:
lipid extracts of plankton or algae, such as Laminaria ochroleuca, which are rich in eicosapentaenoic acid and docosahexaenoic acid,
vegetable oils containing triglycerides rich in alpha-linolenic acid, such as rapeseed, soy or linseed oil,
fish oils rich in alpha-linolenic, eicosapentaenoic and docosahexaenoic acids, and
products obtained by synthetic or biosynthetic chemistry of the mono-, di- or triglyceride type, as well as phospholipids and glycolipids whose fatty acid composition is between 10 and 100% of alpha-linolenic, eicosapentaenoic and docosahexaenoic acids.

The constituent elements of this dermal and/or cosmetic galenic base make it possible to render it neutral towards the skin and to guarantee that it is perfectly harmless on the skin, i.e. they enable it to respect the integrity of the skin and render it particularly suitable for reactive skin. These results are obtained by improving and/or increasing the tolerability of both the aqueous phase and the fatty phase, by improving the cell viability and by the non-reactivity of the components, i.e. their neutrality towards the active ingredients and the ingredients conventionally used in cosmetics and dermo-cosmetics.

The constituent elements of a base according to the invention also make it possible to maintain and restore cutaneous homeostasis, including for skin in a pathological condition, and, when active ingredients are incorporated, promote their reception by the skin.

The dermal and/or cosmetic galenic bases of the present invention preserve their tolerability level, irrespective of the active ingredient(s) incorporated and carried, and protect from the cutaneous cellular degradations associated with environmental factors.

By virtue of their respective content of polyols or liporegulatory substances, the dermal and/or cosmetic galenic bases according to the invention, namely the aqueous phase and/or the fatty phase, are useful for guaranteeing the general ecosystem of the skin.

Preferably, a dermal and/or cosmetic galenic base according to the invention is characterized in that the total polyol content is between 0.1 and 40% of the total weight of the aqueous phase.

If the galenic base contains a fatty phase according to the invention, the total liposoluble polyol content is between 0.01 and 10% of the total weight of the fatty phase.

If the galenic base contains a fatty phase according to the invention, the total content of liporegulatory substances is between 0.01 and 100% of the total weight of the fatty phase.

‘Polyol’ is understood as meaning a hydrocarbon organic compound having several hydroxyl groups.

‘Liposoluble polyol’ is understood as meaning a polyol as defined above which has a significant solubility in a fatty phase, or a polyol chemically modified by the grafting or addition of a liposoluble chain or by the polymerization of several polyol units.

‘Ose’ is understood as meaning a carbohydrate containing from 3 to 8 carbon atoms, all of which carry an oxygen-containing characteristic functional group, namely a hydroxyl, ketone and/or aldehyde group.

‘Oside’ is understood as meaning a compound of the carbohydrate family which is a product resulting from the condensation, with the elimination of water, of molecules of oses or ose derivatives bonded together by glycosidic linkages.

‘Ose reduction product’ is understood as meaning a linear polyol obtained by reduction of the aldehyde functional group which cyclizes an ose.

‘Liporegulatory substance’ is understood as meaning a lipid substance rich in polyunsaturated fatty acids of the omega 3 and/or omega 6 type (especially alpha- and gamma-linolenic acid, eicosapentaenoic acid, docosa-hexaenoic acid) which, by becoming incorporated in the cutaneous molecular structures (cell membrane, epidermal intercellular cement), make it possible significantly to lower the reactivity threshold of the skin.

The present invention further relates to the use of at least one polyol selected from the group comprising osides, oses and ose reduction products, in the aqueous phase of a dermal and/or cosmetic galenic base, for improving its tolerability and optimizing the effects of the active ingredients.

In one particular embodiment according to the invention, the polyol is selected from the group of oses comprising glucose, rhamnose, xylose, mannose and fructose.

In another embodiment, the polyol is selected from the group of ose reduction products comprising mannitol and xylitol.

In another embodiment, the polyol is selected from the group of osides such as fructooligosaccharide, the trisaccharide polymer of α-L-fucose-1->3-α-D-galactose-1->3-α-D-galacturonic acid, hyaluronic acid, chondroitin sulfate, cyclodextrins, galactoarabinan and insulin.

The present invention will now be explained from the experimental point of view.

Demonstration of the Improvement in Tolerability

The properties of the improvement in tolerability by the polyols as defined above were verified by a test that made it possible to demonstrate the non-degradation of the allostimulating function of human epidermal Langerhans cells.

The polyols were dissolved at a concentration of 2 mg/ml in a support.

The supports used, namely xylitol, rhamnose, mannitol and fructooligosaccharide, were tested in a mixed lympho-epidermal culture, separately or together, at final concentrations of 1 and 10%.

The test was conducted according to the protocol described in “Human in vitro T cell sensitization using hapten-modified epidermal Langerhans cells”, Advances in Experimental Medicine and Biology, 1993, 209, p. 212, C. Moulon et al.

Preliminary viability assays on the Langerhans cells after 18 hours of incubation in the presence of the different products did not show any toxic effect at the doses used.

The results of three experiments carried out with cells originating from different donors show that, at doses of 1 or 10%, the different products do not significantly modify the allostimulating function of Langerhans cells. Only a slight decrease in this function is observed in one experiment out of 3 when the polysaccharides are added together to the mixed lympho-epidermal culture at a dose of 10%. Interleukin-10 (IL10), known for its immunosuppressive effect, is used as control.

The results show that, under normal conditions, the different polyol supports tested do not modify the allostimulating function of human epidermal Langerhans cells.

Desensitizing Activity

The desensitizing activity of the lipid extract of Laminaria ochroleuca is observed by the lowering of the reactivity threshold caused by an irritant molecule, namely DNFB (dinitrofluorobenzene).

This activity of the lipid extract of Laminaria ochroleuca was verified by dissolving the lipid extract of Laminaria ochroleuca at a concentration of 2% in the fatty phase of a dermal and/or cosmetic galenic base according to the invention. The composition obtained was applied to an experimental subject at a rate of 12.5 μl of cream in the morning and evening for 3 days.

After 3 days of application, an irritant dose of DNFB (dinitrofluorobenzene) (0.4%) is applied and the edema is measured 3, 6, 9 and 24 hours after application. The results obtained show a decrease in the edema on the skin after application of the base containing the lipid extract of Laminaria ochroleuca, compared with application of the same dose of DNFB without this base.

Cell Viability

The cell viability of fibroblasts is assessed by the WST-1(*) conversion technique, which consists in evaluating the activity of the succinate/tetrazolium reductase mitochondrial system of living cells.

WST-1 (Boehringer/Roche) is reduced to a colored precipitate of formazan. The cell viability is determined by the spectrophotometric reading at 450 nm. The intensity of the optical density is proportional to the number of living cells.

(*): WST-1 tetrazolium salt: 4-(3-(4-iodophenyl)-2-(4-nitrophenyl)-2H-5-tetrazolio)-1,3-benzenedisulfonate

Inoculation

The fibroblasts are inoculated into 96-well microplates at a rate of 10,000 cells per well in 200 μl of standard DMEM (SIGMA) enriched with growth factors (10% FCS). The plates are incubated for 10 min and then for 2 to 4 h at 37° C. in a humid atmosphere containing 6% of CO₂.

Range of Concentrations Tested

The different concentrations to be tested are prepared from a stock solution of the polyols according to the invention at 0.1 g/l to 10 g/l in water.

Treatment

After removal of the DMEM, the different dilutions of product are brought into contact with the cells. The media are not renewed during the experiment. Each point is performed in triplicate.

The cytotoxicity (WST-1 test) is measured after contact times of 10 min and 2 and 4 h.

The optical density is read with an ELISA microplate reader at 450 nm.

| | | | | | | Distilled |
|---|---|---|---|---|---|---|
| | | | 0.1 g/l | 1 g/l | 10 g/l | water |
| Viability | 10 | min | 100 | 100 | 100 | 5 |
| | 2 | h | 98 | 95 | 101 | 0 |
| | 4 | h | 65 | 70 | 85 | 0 |

The results show an increase in viability for solutions containing the polyols incorporated in the aqueous phase of the dermal and/or cosmetic galenic base according to the invention, even after 4 hours.

Study of the Skin Tolerance and Sensitizing Power

The study is performed by the method of Marzulli-Maibach on 50 volunteers on whom patches are applied to the back at a d=homolateral site and a controlateral site.

Two formulations, namely “RO4FF17” and “RO4FF18”, were tested, the reference “RO4FF17” representing a galenic base of the prior art and the reference “R04FF18” representing this same galenic base modified according to the invention.

R04FF17: galenic base of the prior art

| | |
|---|---|
| Mineral oil | 12.0% |
| PEG-8 stearate | 6.00% |
| Glyceryl stearate | 2.00% |
| PEG-100 stearate glyceryl stearate | 2.00% |
| Cetyl alcohol | 2.00% |
| Stearic acid | 2.00% |
| Shea butter | 1.00% |
| Sorbitan sesquioleate | 0.50% |
| Phenoxyethanol | 0.30% |
| Propyl paraben | 0.15% |
| Methyl paraben | 0.15% |
| Butyl paraben | 0.10% |
| Allantoin | 0.10% |
| Triethanolamine | 0.34% |
| Ethyl paraben | 0.07% |
| Water | qsp |

R04FF18: galenic base of the prior art modified according to the invention

| | |
|---|---|
| Mannitol | 1 |
| Rhamnose | 0.5 |
| Xylitol | 5 |
| Fructooligosaccharide | 5 |

1—Determination of the Sensitizing Power

The product is considered to be sensitizing if a subject at least presents all the following signs, irrespective of the side on which it occurs:
erythema with or without edema
pruritus
vesicle

No significant manifestation of intolerance was observed by the investigator for the two bases tested, under the study conditions.

2—Determination of the Irritation Index Z

Z is calculated on the basis of the erythema parameter (including edema) and the desquamation parameter according to the following formula:

$$Z = \frac{\begin{matrix}\sum ( erythema + desquamation scores ) product - \\ \sum ( erythema + desquamation scores ) control\end{matrix}}{number of subjects \times number of readings}$$

The erythema and desquamation scores are calculated as follows:
erythema score=erythema dimensions×number of occurrences
desquamation score=desquamation dimensions×number of occurrences

Z is calculated for the homolateral zone and the controlateral zone. The highest value is taken into account for the interpretation.

The Z interpretation grid is as follows:

| Class | Value of Z | Irritating potential |
|---|---|---|
| 1 | Z < 0.01 | practically zero |
| 2 | 0.01 ≦ Z ≦ 0.04 | very weak |
| 3 | 0.05 ≦ Z ≦ 0.09 | weak |
| 4 | Z < 0.10 | moderate |

The product R04FF17 has an irritation index Z of 0.05. The product R04FF18 has an index Z of 0.00.

Modification of the galenic base according to the invention improves its score to a very advantageous extent.

According to the invention, the dermal and/or cosmetic galenic base has a pH similar to that of the skin, between 4 and 7, and is presented in a form suitable for dermal application.

Said bases are presented especially in the form of aqueous or oily solutions or alcoholic solutions, dispersions, gels, emulsions obtained by dispersing a fatty phase in an aqueous phase, or vice-versa, suspensions or emulsions. These preparations are formulated according to protocols normally used in the field of cosmetic and/or dermo-cosmetic formulation.

In particular, the fatty phase can comprise any fat conventionally used in the field of application envisaged. Fats which may be mentioned especially are silicone-based fats such as silicone oils, gums and waxes, as well as non-silicone-based fats such as oils and waxes of vegetable, mineral, animal and/or synthetic origin. The oils can optionally be volatile or non-volatile.

The present invention therefore further relates to a cosmetic and/or dermo-cosmetic composition, characterized in that it comprises a dermal and/or cosmetic galenic base according to the invention as defined above.

These cosmetic bases will conventionally be used for preparing protective, treating or care creams, body milks, lotions, skin care or cleaning gels, make-up products (e.g. foundation, mascara, lipstick) and hair cleaning and care products (shampoo, lotion, cream).

In known manner in the cosmetic and/or dermo-cosmetic field, the galenic bases according to the invention can contain the hydrophilic and/or lipophilic active ingredients required for the intended activity, and the adjuvants normally used in the cosmetic, dermo-cosmetic or dermatological field. Examples of these adjuvants, without implying a limitation, are hydrophilic and/or lipophilic gelling agents, preservatives, antioxidants, solvents, perfumes, fillers and colorants. The amounts of these various adjuvants are those conventionally used in the fields in question, e.g. from 0.01 to 20% of the total weight of the composition.

A dermal and/or cosmetic galenic base according to the invention is now illustrated by means of the Formulation Examples given below, the compositions being by weight.

### EXAMPLE 1

### Dermal and/or Cosmetic Galenic Base for a Cream Intended for Delicate Skin

A - Fatty phase

| | |
|---|---|
| Arachidyl alcohol/behenyl alcohol/ | 1% |
| arachidyl glucoside | |
| Glycerol stearate | 5% |
| Lipid extract of Laminaria ochroleuca | 1% |
| Squalane | 15% |
| Cetyl alcohol | 2% |

B - Aqueous phase

| | |
|---|---|
| Water | qsp 100% |
| Glycerol | 2.0% |
| Hexylene glycol | 3.0% |
| Xanthan gum | 0.5% |
| Preservatives | qs |
| Carbomer | 0.35% |

C - Ingredients added to the emulsion
at a temperature below 50° C.

| | |
|---|---|
| Mannitol | 0.75% |
| Fructooligosaccharide | 5.0% |
| Rhamnose | 0.3% |
| Xylitol | 1.0% |
| Water | 2% |
| Water | 1.5% |
| NaOH | 0.35% |

### EXAMPLE 2

### Dermal and/or Cosmetic Galenic Base for a Cream Intended for Normal Skin

A - Fatty phase

| | |
|---|---|
| Ceteareth-2 | 3.5% |
| Ceteareth-21 | 2 to 4% |
| Wheatgerm oil | 3% |
| Cyclomethicone | 7% |
| Octyl palmitate | 8% |
| Lipid extract of Laminaria ochroleuca | 0.01% |

B - Aqueous phase

| | |
|---|---|
| Water | qsp 100% |
| Glycerol | 7.0% |
| Hexylene glycol | 3.0% |
| Preservatives | qs |

C - Ingredients added to the emulsion
at a temperature below 50° C.

| | |
|---|---|
| PCANa | 0.5% |
| Mannitol | 0.5% |
| Fructooligosaccharide | 3.0% |
| Rhamnose | 0.1% |
| Xylitol | 2.0% |
| Sodium hyaluronate | 0.1% |
| Water | 5% |
| Tocopherol | 0.05% |
| Vitamin A palmitate | 0.1% |
| Phospholipids | 0.5% |
| Ceramides 3 | 0.1% |
| Polyacrylamide & C₁₄₋₁₃ isoparaffin & | 2 to 3.5% |
| laureth-7 | |

### EXAMPLE 3

### Dermal and/or Cosmetic Galenic Base for a Cream and Milk Intended for Skin Exposed to Sunlight

A - Fatty phase

| | |
|---|---|
| Glycerol monostearate | 2% |
| PEG-100 stearate | 3% |
| C12-C15 alkyl benzoate | 10% |
| Lipid extract of Laminaria ochroleuca | 5% |
| Dimethicone | 5% |
| Tocopherol acetate | 1% |
| Octyl-triazone (Uvinul T150) | 1.5% |
| Butylmethoxydibenzoylmethane | 2.0% |
| (Eusolex 9020) | |
| Cetostearyl alcohol | 1% |

B - Aqueous phase

| | |
|---|---|
| Water | qsp 100% |
| Preservatives | 0.6% |
| Glycerol | 7% |
| Hexylene glycol | 3.0% |
| Carbomer | 0.5% |
| Tetrasodium EDTA | 0.2% |

C - Ingredients added to the emulsion
at a temperature below 50° C.

| | |
|---|---|
| Serine | 0.2% |
| Mannitol | 0.5% |
| Fructooligosaccharide | 3.0% |
| Rhamnose | 0.1% |
| Xylitol | 2.0% |
| Sodium hyaluronate | 0.1% |
| Water | 5% |
| NaOH | 0.5% |
| Perfume | qs |

### EXAMPLE 4

### Dermal and/or Cosmetic Galenic Base for a Milk or Cream Intended for Skin Rendered Delicate by an Irritant

A - Fatty phase

| | |
|---|---|
| Lipid extract of Laminaria ochroleuca | 5% |
| squalane | 5% |
| Cetyl alcohol | 2% |
| Dimethicone | 5% |
| Octyl palmitate | 5% |

B - Aqueous phase

| | |
|---|---|
| Butylene glycol | 0.5-4% |
| Water | qsp 100% |
| Glycerol | 2.0% |
| Hexylene glycol | 3.0% |
| Biosaccharide gum 2 | 1.0% |
| Xanthan gum | 0.5% |
| Preservatives | qs |

C - Ingredients added to the emulsion
at a temperature below 50° C.

| | |
|---|---|
| Glycyrrhetinic acid | 0.1-1% |
| Mannitol | 0.5% |
| Fructooligosaccharide | 3.0% |
| Rhamnose | 0.1% |
| Xylitol | 2.0% |
| Water | 2.3% |
| Tocopherol acetate | 0.1 to 1% |
| Pyridoxine | 0.01 to 0.05% |
| Vitamin A palmitate | 0.01 to 1% |
| d-Panthenol | 0.1 to 1% |
| Citric acid | 0.1 to 0.5% |
| Zinc gluconate | 0.1 to 1% |
| Trisodium citrate | 1 to 2.5% |
| L-fucose | 0.01 to 1% |
| Water | 5% |

### EXAMPLE 5

### Dermal and/or Cosmetic Galenic Base for a Cream or Milk for Skin Having Deficiencies Associated With Skin Ageing

A - Fatty phase

| | |
|---|---|
| Lipid extract of Laminaria ochroleuca | 0.5% |
| Glyceryl stearate | 1 to 5% |
| Stearic acid | 1 to 5% |
| Isononyl isononanoate | 1 to 15% |

B - Aqueous phase

| | |
|---|---|
| Water | qsp 100% |
| Glycerol | 3.0% |
| Xanthan gum | 0.5% |
| Preservatives | qs |

C - Ingredients added to the emulsion
at a temperature below 50° C.

| | |
|---|---|
| Mannitol | 0.5% |
| Fructooligosaccharide | 3.0% |
| Rhamnose | 0.1% |
| Xylitol | 2.0% |
| Water | 7.8% |
| Pyridoxine | 0.01 to 0.05% |
| Citric acid | 0.1 to 0.5% |
| Zinc gluconate | 0.1 to 1% |
| Trisodium citrate | 1 to 2.5% |
| Water | 2% |
| d-Panthenol | 0.1 to 1% |
| Vitamin A palmitate | 0.01 to 1% |
| Ascorbyl palmitate | 0.01 to 0.1% |
| Tocopherol acetate | 0.1 to 1% |
| L-fucose | 0.01 to 1% |
| Lactoferrin/lactoperoxidase | 0.01 to 1% |
| Water | 2% |

### EXAMPLE 6

### Dermal and/or Cosmetic Galenic Base for a Cream and Milk for Hyperseborrheic Skin or Skin With a Tendency to Greasiness

A - Fatty phase

| | |
|---|---|
| Ceteareth-2 | 3.5% |
| Ceteareth-21 | 2 to 4% |
| Lipid extract of Laminaria ochroleuca | 5% |
| Squalane | 5% |
| Cetyl alcohol | 2% |

B - Aqueous phase

| | |
|---|---|
| Water | qsp 100% |
| Dipropylene glycol | 1-8% |
| Dimethicone copolyol | 0.1-5% |
| Disodium EDTA | 0.05-0.5% |
| Preservatives | qs |

C - Ingredients added to the emulsion
at a temperature below 50° C.

| | |
|---|---|
| Salicylic acid | 0.1-0.5% |
| Zinc gluconate | 0.1-1% |
| Water | 3% |
| Ascorbyl palmitate | 0.01 to 0.1% |
| Tocopherol acetate | 0.1 to 1% |
| Vitamin A palmitate | 0.01 to 1% |
| d-Panthenol | 0.1 to 1% |
| Pyridoxine | 0.01 to 0.05% |
| Citric acid | 0.1-0.5% |
| Trisodium citrate | 1 to 2.5% |
| Mannitol | 0.5% |
| Fructooligosaccharide | 3.0% |
| Rhamnose | 0.1% |
| Xylitol | 2.0% |
| Rhamnose | 0.1 to 1% |
| L-fucose | 0.01 to 1% |
| Superoxide dismutase | 0.01 to 1% |
| Water | 4% |

### EXAMPLE 7

### Dermal and/or Cosmetic Galenic Base for an Isotonic Lotion

| | |
|---|---|
| Hexylene glycol | 4% |
| d-Panthenol | 0.1% |
| Mannitol | 0.02% |
| Fructooligosaccharide | 2.0% |
| Rhamnose | 0.01% |
| Xylitol | 0.50% |
| Trimethylglycine | 2% |
| Preservatives | qs |
| Water | qsp 100% |

### EXAMPLE 8

### Dermal and/or Cosmetic Galenic Base for a Make-Up Removing Lotion

A - Aqueous phase

| | |
|---|---|
| Polysorbate 20 | 1.0% |
| Caprylyl/capryl glucoside (Oramix | 2.0% |
| CG110) | |
| Lipid extract of Laminaria | 0.1% |
| ochroleuca | |
| PEG-7 glyceryl cocoate | 0.5% |
| Hexylene glycol | 4-5% |
| d-Panthenol | 0.1% |
| Mannitol | 0.02% |
| Fructooligosaccharide | 1.0% |
| Rhamnose | 0.01% |
| Xylitol | 0.50% |
| Preservatives | qs |
| Water | qsp 100% |

### EXAMPLE 9

### Dermal and/or Cosmetic Galenic Base for an Oil for Skin Rendered Delicate by Irritants

| | |
|---|---|
| Ethylhexyl palmitate | 45% |
| Cyclomethicone | 30% |
| Lipid extract of Laminaria ochroleuca | 10% |
| Tocopheryl acetate | 0.5% |
| Dipropylene glycol | 0.5% |
| Trilinolein | 0.1% |
| Trilinolenin | 0.1% |
| Soy oil | qsp 100% |

1-20. (canceled)

21. A dermal and/or cosmetic galenic base whose aqueous phase contains at least two polyols each selected from the group comprising osides, oses and ose reduction products, characterized in that at least two of these polyols are selected from the group of ose reduction products comprising mannitol and xylitol, and in that at least one polyol is selected from the group of oses comprising glucose, rhamnose, xylose, mannose and fructose.

22. The dermal and/or cosmetic galenic base as claimed in claim 21, wherein one polyol selected from the group of oses is rhamnose.

23. The dermal and/or cosmetic galenic base as claimed in claim 21, wherein one polyol is selected from the group of osides comprising fructooligosaccharides, the trisaccharide polymer of α-L-fucose-1->3-α-D-galactose-1->3-α-D-galacturonic acid, hyaluronic acid, chondroitin sulfate, cyclodextrins, galactoarabinan and inulin.

24. A dermal and/or cosmetic galenic base as claimed in claim 21 whose fatty phase contains at least two liposoluble polyols each selected from the group comprising Rhamnosoft®, cetearyl glucoside, mannitan laurate and glucose glutamate.

25. The dermal and/or cosmetic galenic base as claimed in claim 21, wherein it also contains a fatty phase comprising a substance selected from liporegulatory substances.

26. The dermal and/or cosmetic galenic base as claimed in claim 25, wherein the substance selected from liporegulatory substances is a lipid extract of Laminaria ochroleuca which is rich in eicosapentaenoic acid and docosahexaenoic acid.

27. The dermal and/or cosmetic galenic base as claimed in claim 25, wherein the substance selected from liporegulatory substances is soy oil.

28. The dermal and/or cosmetic galenic base as claimed in claim 25, wherein the substance selected from liporegulatory substances is linseed oil.

29. The dermal and/or cosmetic galenic base as claimed in claim 25, wherein the substance selected from liporegulatory substances is rapeseed oil.

30. The dermal and/or cosmetic galenic base as claimed in claim 25, wherein the substance selected from liporegulatory substances is a fish oil rich in alpha-linolenic, eicosapentaenoic and docosahexaenoic acids.

31. The dermal and/or cosmetic galenic base as claimed in claim 25, wherein the substance selected from liporegulatory substances is a product obtained by synthetic or biosynthetic chemistry of the mono-, di- or triglyceride type, or a phospholipid or glycolipid whose fatty acid composition is between 10 and 100% of alpha-linolenic, eicosapentaenoic and docosahexaenoic acids.

32. A method of cosmetic treatment, comprising applying to skin a cosmetic composition comprising a dermal and/or cosmetic galenic base according to claim 1.

33. The method as claimed in claim 32, wherein the polyol selected from the group of oses is rhamnose.

34. The method as claimed in claim 32, wherein the polyol is selected from the group of osides comprising fructooligosaccharides, the trisaccharide polymer of α-L-fucose-1->3-α-D-galactose-1->3-α-D-galacturonic acid, hyaluronic acid, chondroitin sulfate, cyclodextrins, galactoarabinan and inulin.

35. A method of cosmetic treatment, comprising applying to skin at least two liposoluble polyols each selected from the group consisting of Rhamnosoft®, cetearyl glucoside, mannitan laurate and glucose glutamate, in the fatty phase of a dermal and/or cosmetic galenic base.

36. The dermal and/or cosmetic galenic base as claimed in claim 21, wherein the total polyol content is between 0.1 and 40% of the total weight of the aqueous phase.

37. The dermal and/or cosmetic galenic base as claimed in claim 24, wherein the total content of liposoluble polyols is between 0.01 and 10% of the total weight of the fatty phase.

38. The dermal and/or cosmetic galenic base as claimed in claim 24, wherein the total content of liporegulatory substances is between 0.01 and 100% of the total weight of the fatty phase.

39. A cosmetic and/or dermo-cosmetic composition, comprising a base as claimed in claim 21.
